(19) United States
(12) Patent Application Publication
(10) Pub. No.: US 2026/0212995 A1
(43) Pub. Date: Jul. 23, 2026

(54) MEDICAL IMAGING PROTOCOL MAPPING USING ARTIFICIAL INTELLIGENCE

(71) Applicant: GE Precision Healthcare LLC, Waukesha, WI (US)

(72) Inventors: Jithin Anneri Kundacheri, Bangalore (IN); Soumya Ranjan Panigrahi, Bangalore (IN); Sudharshan Venkatareddy, Bangalore (IN); Ann Jose, Bangalore (IN)

(21) Appl. No.: 19/032,800

(22) Filed: Jan. 21, 2025

(51) Int. Cl. G16H 30/00; G16H 20/40

(52) U.S. Cl. CPC G16H 30/00; G16H 20/40

(57) ABSTRACT

Techniques for medical imaging protocol mapping using artificial intelligence are presented. In an example, a method can comprise performing, by a system comprising a processor, a similarity mapping process between pairs of medical imaging protocol data, the pairs respectively comprising planned imaging protocol data in a first format and performed imaging protocol data in a second format. The method further comprises generating, by the system, similarity matrices for the pairs as a result of the similarity mapping process, and training, by the system, a neural network model to estimate similarity scores for the pairs using the similarity matrices as input to the neural network model.

### TECHNICAL FIELD

This disclosure relates generally to medical imaging, and more particularly to medical imaging protocol mapping using artificial intelligence.

### BACKGROUND

Healthcare medical imaging protocol management refers to the systematic development, implementation, monitoring, and optimization of medical imaging protocols across healthcare facilities. It ensures that imaging procedures are performed consistently, safely, and efficiently, adhering to evidence-based guidelines, clinical standards, and patient safety principles.

Protocol management involves creating standardized protocols that define the specific techniques, parameters, and processes used during medical imaging procedures to ensure consistent, high-quality images while optimizing patient safety and resource utilization. These protocols are further tailored to a healthcare facility's equipment, patient population, and clinical workflows, and optimized over time.

In addition to creating and optimizing standardized protocols, protocol management also involves ensuring they are followed properly. In this regard, although standardized protocols are designed to ensure that imaging studies are performed uniformly across departments, facilities, and systems, improving diagnostic reliability, often times, medical imaging technicians deviate from the recommend protocol prescribed for an exam.

Medical imaging technicians may not follow recommended imaging protocols for a variety of reasons, often involving human factors, systemic challenges, and technological barriers. For example, technicians may lack proper training or understanding of the specific protocols. New or updated protocols might not be effectively communicated or taught to the staff. High patient volumes and tight schedules can pressure technicians to prioritize speed over protocol adherence. Differences in imaging equipment across departments or facilities (e.g., older machines) may prevent technicians from strictly following protocols. Protocols optimized for newer technology may not be compatible with older machines. Highly complex or overly detailed protocols can be difficult to implement consistently, leading to errors or shortcuts. Variability in protocols for different patient types (e.g., pediatric vs. adult) can create confusion. In addition, inconsistent protocols between facilities or within departments can cause confusion.

When recommended imaging protocols are not implemented, several issues can arise, impacting patient care, clinical outcomes, operational efficiency, and resource management. Deviating from recommended imaging protocols may result in suboptimal image quality, leading to misdiagnosis or missed findings. Failing to adhere to protocols for dose optimization can expose patients to unnecessary or excessive radiation during imaging procedures like X-ray and computed tomography (CT) scans. In addition, failing to adhere to standardized protocols leads to inconsistencies in imaging practices across facilities, complicating comparison over time or between institutions.

Although various healthcare imaging protocol management solutions exist, these solutions lack techniques for efficiently comparing standardized protocols with actual performed protocols across different medical imaging systems in association with monitoring and assessing adherence to the standardized protocols. This is because different medical imaging systems often employ different terminology, identifiers and data formats to describe the actual performed protocols relative to that used for the standardized protocols.

### SUMMARY

The following presents a simplified summary of the specification in order to provide a basic understanding of some aspects of the specification. This summary is not an extensive overview of the specification. It is intended to neither identify key or critical elements of the specification, nor delineate any scope of the particular implementations of the specification or any scope of the claims. Its sole purpose is to present some concepts of the specification in a simplified form as a prelude to the more detailed description that is presented later.

According to an embodiment, a system includes at least one memory that stores computer-executable components, and at least one processor that executes the computer-executable components stored in the at least one memory. The computer-executable components can comprise a similarity mapping component that performs a similarity mapping process between pairs of medical imaging protocol data, the pairs respectively comprising planned imaging protocol data in a first format and performed imaging protocol data in a second format, and generates similarity matrices for the pairs as a result of the similarity mapping process. The computer-executable components further comprise a training component that trains a neural network model to estimate similarity scores for the pairs using the similarity matrices as input to the neural network model.

In one or more implementations, the training component trains the neural network model using a supervised machine learning process and ground truth information for respective ones of the pairs identifying ground truth similarity scores.

In some implementations, the similarity mapping process comprises, for each pair, determining measures of similarity between respective parameters of a defined set of parameters included in the planned imaging protocol data and the performed imaging protocol data, and generating a similarity matrix for the pair comprising the measures of similarity. For example, in some implementations (e.g., as applied to medical resonance imaging (MRI)), the defined set of parameters comprises one or more parameters selected from the group consisting of: protocol name, series description name, anatomic region, series duration, field of view, slice thickness, percent phase field of view, number of excitations, acceleration factor, specific absorption rate, echo time, receive coil name, pixel bandwidth, repetition time, echo train length and voxel size.

In an aspect, the determining the measures of similarity comprises determining the measures of similarity based on correspondences and differences between terms of the respective parameters in the first format and the second format.

In some implementations, the computer-executable components further comprise an inferencing component that employs a trained version of the neural network model to estimate similarity scores for new pairs of medical imaging protocol data based on new similarity matrices generated for the new pairs by the similarity mapping component using the similarity mapping process, wherein the new pairs respectively comprise new planned imaging protocol data in the first format and new performed imaging protocol data in the second format.

The computer-executable components can further comprise a performance assessment component that identifies a subset of the new pairs with similarity scores below a threshold similarity score, and determines, for respective pairs of the subset, whether one or more performance metrics for imaging scans that adhere to the new performed imaging protocol data satisfy one or more acceptability criteria. For example, the one or more performance metrics can comprise a quality metric that represents a measure of quality of medical images acquired via the imaging scans. Additionally, or alternatively, the one or more performance metrics can comprise a dose metric that represents a measure of radiation dose associated with the imaging scans.

In some embodiments, elements described in connection with the disclosed systems can be embodied in different forms such as a computer-implemented method, a computer program product, or another form.

For example, in another embodiment, a computer-implemented method, can comprise performing, by a system comprising a processor, a similarity mapping process between pairs of medical imaging protocol data, the pairs respectively comprising planned imaging protocol data in a first format and performed imaging protocol data in a second format. The method can further comprise generating, by the system, similarity matrices for the pairs as a result of the similarity mapping process; and training, by the system, a neural network model to estimate similarity scores for the pairs using the similarity matrices as input to the neural network model.

In another embodiment, a non-transitory machine-readable storage medium can comprise executable instructions that, when executed by a processor, facilitate performance of operations, comprising: performing a similarity mapping process between pairs of medical imaging protocol data, the pairs respectively comprising planned imaging protocol data in a first format and performed imaging protocol data in a second format; generating similarity matrices for the pairs as a result of the similarity mapping process; and training a neural network model to estimate similarity scores for the pairs using the similarity matrices as input to the neural network model.

### BRIEF DESCRIPTION OF THE DRAWINGS

Numerous aspects, implementations, objects and advantages of the present invention will be apparent upon consideration of the following detailed description, taken in conjunction with the accompanying drawings, in which like reference characters refer to like parts throughout, and in which:

FIG. 1 illustrates a high-level block diagram of an example system that facilitates medical imaging protocol mapping using artificial intelligence (AI), in accordance with one or more embodiments described herein;

FIG. 2 illustrates a high-level similarity mapping process in accordance with one or more embodiments described herein;

FIG. 3 illustrates a table comprising an example planned protocol in a first data format and a corresponding performed protocol in a second data format, in accordance with one or more embodiments described herein;

FIG. 4 illustrates an example training process for training a neural network model to estimate similarity scores, in accordance with one or more embodiments described herein;

FIG. 5 illustrates an example runtime process for generating similarity scores for new pairs of planned and performed protocol data, in accordance with one or more embodiments described herein;

FIG. 6 illustrates an example protocol management system in accordance with one or more embodiments described herein;

FIG. 7 illustrates an example computer-implemented method for medical imaging protocol mapping using AI, in accordance with one or more embodiments described herein;

FIG. 8 illustrates another example computer-implemented method for medical imaging protocol mapping using AI, in accordance with one or more embodiments described herein;

FIG. 9 illustrates an example computer-implemented method for monitoring adherence to standardized medical protocols, in accordance with one or more embodiments described herein;

FIG. 10 illustrates another example computer-implemented method for monitoring adherence to standardized medical protocols, in accordance with one or more embodiments described herein;

FIG. 11 is a schematic block diagram illustrating a suitable operating environment; and

FIG. 12 is a schematic block diagram of a sample-computing environment.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

As described in the Background Section, although various healthcare imaging protocol management solutions exist, these solutions lack techniques for efficiently comparing standardized protocols with actual performed protocols across different medical imaging systems in association with monitoring and assessing adherence to the standardized protocols. This is because different medical imaging systems often employ different terminology, identifiers and data formats to describe the actual performed protocols relative to that used for the standardized protocols.

With this context in mind, the disclosed subject matter is directed to systems, computer-implemented methods, apparatus and/or computer program products that facilitate mapping medical imaging protocols using artificial intelligence. In one or more embodiments, the disclosed techniques provide for mapping planned medical imaging protocols to performed medical imaging protocols, wherein the planned medical imaging protocols are provided in a first data format and wherein the performed medical imaging protocols are provided in one or more second data formats that are different from the first data format.

As used herein, a “planned imaging protocol” refers to a standardized imaging protocol or a recommended imaging protocol prescribed for a medical imaging exam. The terms “planned imaging protocol,” “planned medical imaging protocol,” “planned protocol,” and the like are used herein interchangeably. When a medical imaging exam is order for a patient, a radiologist and/or automated protocol recommendation software defines the planned protocol for the exam which is distributed to the imaging facility scheduled for the exam (and/or the imaging device and technician operating the imaging device for the exam). The exam is then performed at the imaging facility and the actual protocol used for the performance of the exam is referred to herein as the “performed protocol.” The terms “performed imaging protocol,” “performed medical imaging protocol,” “performed protocol,” and the like are used herein interchangeably. As noted in the Background section, the performed protocol used for the exam may vary from the planned protocol for a variety of reasons.

The performed protocol is recorded with the medical imaging scan data acquired via the exam, typically using a different format relative to the format of the planned protocol data. For example, the data format, terminology and identifiers used for the planned protocol may correspond to a standardized format, while that used by the entity to define the performed protocol (e.g., the medical facility, the imaging device/system, and/or technician used to perform the exam) is often a different data format and employs different terminology and/or identifiers. In addition, different entities (e.g., different medical imaging facilities, imaging devices, and/or technicians) may use different data formats, terminology and identifiers to record the performed protocols.

In various embodiments, the disclosed techniques combine a rules-based technique with an artificial intelligence (AI) technique to enable automatically mapping and comparing planned imaging protocols with their corresponding performed imaging protocols, wherein the planned imaging protocols are provided in a first data format and the performed imaging protocols are provided in one or more second data formats.

The rule-based technique involves performing a similarity mapping process between pairs of medical imaging protocol data and generating similarity matrices for the pairs as a result of the similarity mapping process, wherein the pairs respectively comprise planned imaging protocol data in a first format and performed imaging protocol data in one or more second data formats. In an embodiment, the similarity mapping process comprises, for each pair, determining measures of similarity between respective parameters of a defined set of parameters included in the planned imaging protocol data and the performed imaging protocol data, and generating a similarity matrix for the pair comprising the measures of similarity. To this end, the determining the measures of similarity comprises determining the measures of similarity based on correspondences and differences between terms (e.g., textual terms) of the respective parameters in the first format and the second format.

The defined set of parameters can vary based on the modality of the imaging exam and the particular protocol. For example, as applied to MRI, protocol name, series description name, anatomic region, series duration, field of view, slice thickness, percent phase field of view, number of excitations, acceleration factor, specific absorption rate, echo time, receive coil name, pixel bandwidth, repetition time, echo train length and voxel size.

The AI technique involves training a neural network model to estimate similarity scores for the pairs using the similarity matrices as input to the neural network model. In one or more embodiments, the training comprises training the neural network model using a supervised machine learning process and ground truth information for respective ones of the pairs identifying ground truth similarity scores. In this manner, as opposed to training the neural network model to assess similarity between raw text, the neural network model assesses similarity based on a similarity matrix that represents the semantic relationships between the respective planned and performed protocols.

Once trained, the disclosed techniques employ the trained version of the neural network model to estimate similarity scores for new pairs of medical imaging protocol data based on new similarity matrices generated for the new pairs using the similarity mapping process, wherein the new pairs respectively comprise new planned imaging protocol data in the first data format and new performed imaging protocol data a second data format.

To this end, the similarity scores output by the trained version of the neural network model indicate how similar (or deviant) respective performed protocols used for actual imagining exams are relative to their corresponding planned protocols. In various embodiments, these similarity scores can be used to automatically identify imaging exams and corresponding imaging systems (and/or imaging devices, technicians, etc.) that deviate from the recommended protocols, enabling efficient remediation. The disclosed techniques can further enable root cause analysis as to the basis why and its impact on resulting image quality, radiation dose exposure, and other performance valuation metrics.

By employing the trained version of the neural network model to measure the similarity between planned verses performed medical imaging protocols, the disclosed system provides a feedback loop that fosters continuous improvement in medical imaging practices, ultimately benefiting patients and healthcare providers. In this regard, developing an AI model that automatically determines the similarity between planned and performed medical imaging protocols offers numerous technical advantages that enhance healthcare delivery, streamline operations, and improve diagnostic outcomes. For example, AI can process and analyze large volumes of imaging protocol data far more quickly than manual reviews, enabling real-time feedback. In addition, the trained version of the neural network model can monitor multiple imaging machines, facilities, or even healthcare networks simultaneously, ensuring consistency at scale. The AI model further minimizes human errors in detecting discrepancies between planned and performed protocols and ensures uniformity across diverse clinical environments, improving diagnostic consistency and reproducibility. In addition, AI can provide instant alerts to technologists when deviations from the planned protocol are detected, allowing for on-the-spot corrections and proactive intervention by prevents protocol mismatches before imaging is completed, reducing the need for repeat scans. Further, the trained version of the neural network model can detect subtle differences between protocols that might not be apparent through manual review and provides unbiased evaluations of protocol adherence, improving quality control measures. Automating protocol similarity checks also frees up radiologists and technologists to focus on more critical tasks, improving productivity. By leveraging AI to automate the similarity assessment between planned and performed imaging protocols, healthcare providers can achieve a high level of precision, efficiency, and quality assurance while optimizing patient safety and outcomes.

As used herein, the term medical imaging device refers to a machine used in healthcare to create visual representations (i.e., medical images) of the inside of a body. Various different types of medical imaging devices exist that vary with respect to modality, that is the specific type of imaging technique or method used to create images of the body. Common types of medical imaging devices include, but are not limited to, X-ray devices, digital radiography (DX) X-ray devices, X-ray angiography (XA) devices, panoramic X-ray (PX) images, computerized tomography (CT) device, mammography (MG) devices (including tomosynthesis devices), magnetic resonance imaging (MRI) device, ultrasound (US) device, color flow doppler (CD) devices, position emission tomography (PET) devices, single-photon emissions computed tomography (SPECT) device, nuclear medicine (NM) devices, and the like. Most medical imaging devices include or incorporate computer processing technology for several critical functions, ranging from image acquisition to advanced processing and analysis. To this end, the term medical image device refers to the entirety of the machine or machines (e.g., including one or more computers and associated processing technology/software) used by the medical image device to acquire raw image data and convert the raw image data into usable medical images.

The terms “medical image,” “medical image data,” and the like are used to refer to image data that depicts one or more anatomical regions of a patient. Reference to a medical image or medical image data herein can include any type of medical image associated with various types of medical image acquisition/capture modalities. For example, medical images can include (but are not limited to): radiation therapy (RT) images, XR images, DX-ray images, XA images, PX images, CT images, MG images, MRI images, US images, CD images, PET images, SPECT images, NM images, and the like. Medical images can also include synthetic versions of native medical images such as augmented, modified or enhanced versions of native medical images, augmented versions of native medical images, and the like generated using one or more image processing techniques. In some embodiments, the term “image data” can include the raw measurement data (or simulated measurement data) used to generate a medical image (e.g., the raw measurement data captured via the medical image acquisition process).

The terms “algorithm” and “model” are used herein interchangeably unless context warrants particular distinction amongst the terms. The terms “artificial intelligence (AI) model” and “machine learning (ML) model” are used herein interchangeably unless context warrants particular distinction amongst the terms. Reference to an AI or ML model herein can include any type of AI or ML model, including (but not limited to): deep learning (DL) models, neural network models, deep neural network models (DNNs), convolutional neural network models (CNNs), generative adversarial neural network models (GANs), transformer models, and the like. An AI or ML model can include supervised learning models, unsupervised learning models, semi-supervised learning models, combinations thereof, and models employing other types of ML learning techniques. An AI or ML model can include a single model or a group of two or more models (e.g., an ensemble model, chained models, or the like).

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

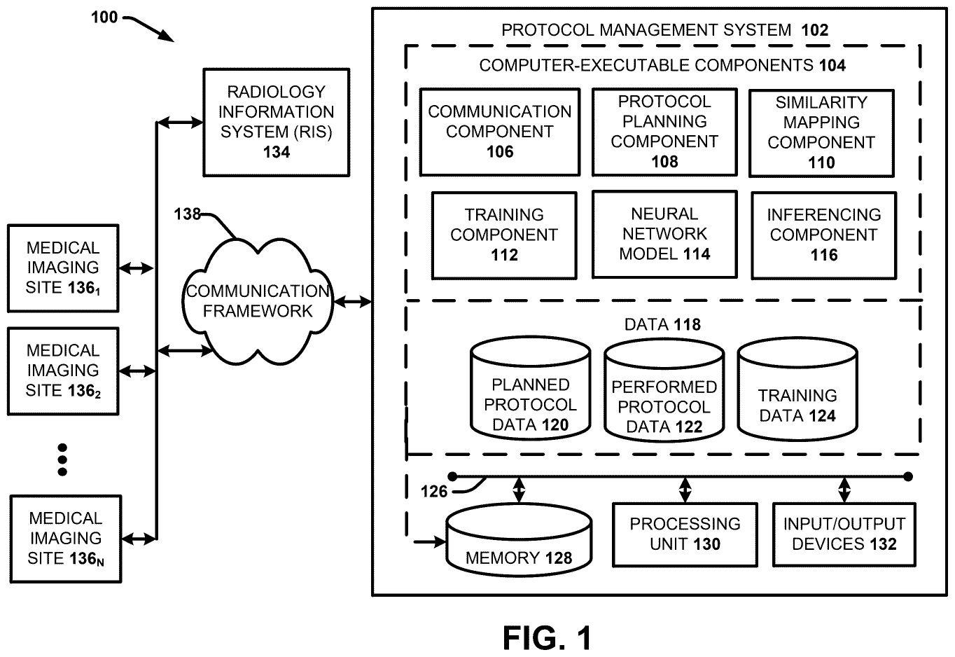

Turning now to the drawings, FIG. 1 illustrates a high-level block diagram of an example system 100 that facilitates medical imaging protocol mapping using AI, in accordance with one or more embodiments. System 100 can include or correspond to one or more computing devices, machines, virtual machines, computer-executable components, datastores, and the like that may communicatively coupled to one another either directly or via one or more wired or wireless communication frameworks. Aspects of the systems, apparatuses or processes explained in this disclosure can constitute computer-executable or machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described.

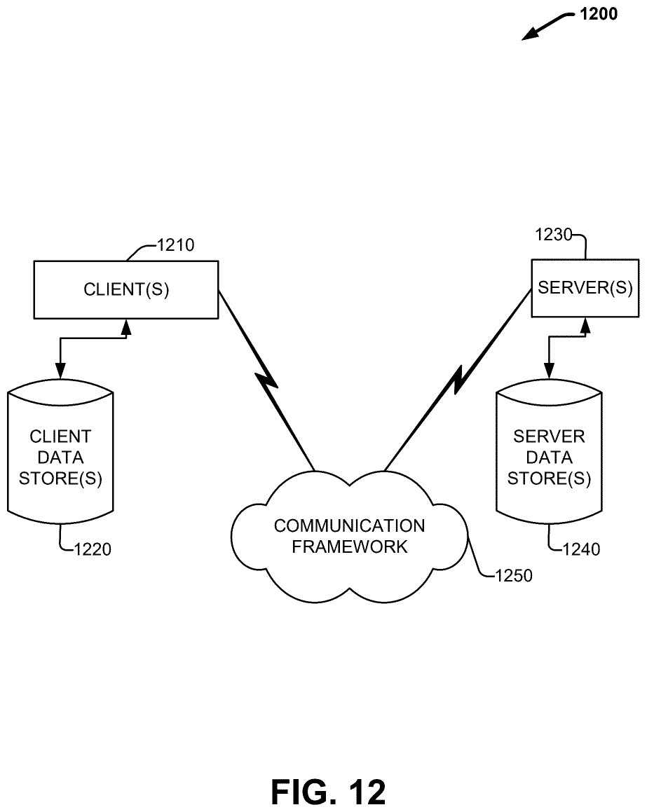

In this regard, system 100 includes protocol management system 102, radiology information system (RIS) 134, and a plurality of medical imaging sites 136_{1-N}, (the number of which (N) can vary), respectively connected to one another via communication framework 138. Communication framework 138 can include or correspond to any existing or future developed wired or wireless communication frameworks (examples of which are described with reference to FIG. 12). Protocol management system 102, RIS 134 and the medical sites 136_{1-N} can respectively include or correspond to one or more computing devices, machines, virtual machines, computer-executable components, and/or datastores.

For example, in various embodiments, protocol management system 102 can include or correspond to a cloud-based medical imaging protocol management system that facilitates systematic development, implementation, monitoring, and optimization of medical imaging protocols across medical imaging sites 136_{1-N}. To this end, the medical imaging sites 136_{1-N} can respectively include or correspond to medical imaging facilities, departments, institutions or the like, that perform medical imaging exams on patients using one or more medical imaging devices. For example, in some implementations, a single medical imaging site of amongst the medical imaging sites 136_{1-N} may provide a variety of different types of medical imaging devices (e.g., with respect to modality, device capabilities, device model, etc.) respectively configured to perform different types of medical imaging exams (e.g., X-ray, CT, MRI, and others). In other implementations, each medical imaging site 136_{1-N} can include or correspond to a single medical imaging device.

In some embodiments, information identifying the respective medical imaging sites 136_{1-N}, the medical imaging devices provided by the respective medical imaging sites 136_{1-N}, capabilities of the imaging devices (e.g., device type, modality, make/model, etc.), and imaging exams performed and ordered or scheduled for performance at the respective medical imaging sites 136_{1-N} can be provided by the RIS 134. A radiology information system (RIS) is a specialized healthcare system used in radiology departments to manage medical imaging records and related data. It plays a crucial role in the workflow of radiology departments, enabling the management of patient data, imaging procedures, results, and administrative tasks.

In various embodiments, RIS 134 corresponds to a centralized RIS that manages medical imaging records and related data associated with all the medical imaging sites 136_{1-N}. For example, in some implementations, RIS 134 can maintain and track information identifying the respective medical imaging sites 136_{1-N}, the medical imaging devices provided by the respective medical imaging sites 136_{1-N}, capabilities of the imaging devices (e.g., device type, modality, make/model, etc.), and imaging exams performed and ordered or scheduled for performance at the respective medical imaging sites 136_{1-N.} In some embodiments, RIS 134 can also include or be integrated with one or more Picture Archiving and Communication Systems (PACS) that store medical image data acquired via respective exams performed at the medical imaging sites 136_{1-N.} The PACS can also store metadata associated with the medical image data, including information identifying the acquisition protocol and corresponding acquisition parameters used. In this regard, the RIS typically handles the administrative and operational aspects of radiology services, while PACS is responsible for storing, retrieving, and viewing medical images. Together, RIS and PACS form the backbone of radiology departments, streamlining both image management and workflow processes.

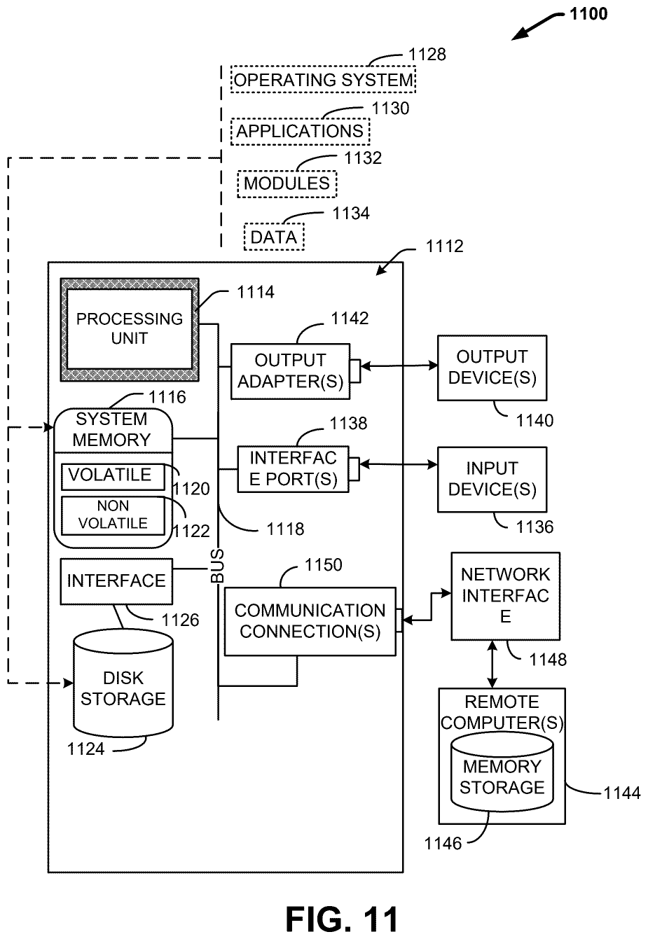

Protocol management system 102 can comprise at least one memory 128 that stores computer-executable components 104, and at least one processor or processing unit 130 that executes the computer-executable components 104 stored in the at least one memory 110. The computer-executable components include, but are not limited to, communication component 106, protocol planning component 108, similarity mapping component 110, training component 112, neural network model 114 and inferencing component 116. Examples of said memory 128 and processing unit 130 as well as other suitable computer or computing-based elements, can be found with reference to FIG. 11 (e.g., system memory 1116 and processing unit 1114 respectively), and can be used in connection with implementing one or more the components shown and described in connection with FIG. 1, or other figures disclosed herein. Memory 128 can also store data 118 that is received by, used by, and/or generated by the protocol management system 102, including but not limited to, planned protocol data 120, performed protocol data 122 and training data 124. Additionally, or alternatively, any information that is used by the computer-executable components 104 can be stored at another network accessible device and accessed by the protocol management system 102 via communication framework 138.

Protocol management system 102 can further include one or more input/output devices 132 to facilitate receiving user input and rendering data to users in association with performing various operations described with respect to the order computer-executable component 104. Suitable examples of the input/output devices 132 are described with reference to FIG. 11 (e.g., input devices 1136 and output devices 1140). Protocol management system 102 can further include a system bus 126 that couples the memory 128, the processing unit 130 and the input/output devices 132 to one another.

Communication component 106 includes or corresponds to the hardware and/or software that enables wired and/or wireless communication between the protocol management system 102 and external systems, devices, machines, databases, etc. (e.g., RIS 134, medical imaging sites 136_{1-N} and others) via communication framework 138.

In accordance with various embodiments, protocol management system 102 facilitates comparing planned medical imaging protocols for imaging exams performed at the imaging sites 136_{1-N} with their corresponding actual performed protocols in association with monitoring and assessing adherence to the planned protocols. As noted above, the planned protocols correspond to standardized and/or recommended protocols to be used for the medical imaging exams while the performed protocols correspond to the actual protocols used for the respective exams. As noted in the Background section, the performed protocol used for the exam may vary from the planned protocol for a variety of reasons.

In this regard, imaging exam orders are prescribed by an ordering physician after evaluating a patient. The imaging exam order typically includes information identifying the patient (e.g., via a unique identifier (ID) such as the patient's name, or another type of anonymous ID), the ordering physician, the clinical indication for the exam or exam (e.g., a detailed explanation of why the imaging is being ordered, such as the known or suspected diagnosis, symptoms, clinical findings, etc.), the specific modality of the exam (e.g., X-ray, CT, MRI, etc.), and the ROI to be imaged (e.g., the specific body part or anatomical region to be imaged). In some cases, the order may also include additional information, such as special instructions (e.g., any particular imaging techniques or acquisition protocols/required, such as with or without contrast, high-resolution imaging, etc.), a level of urgency of the exam, and relevant patient preference and/or constraints associated with performance of the exam (e.g., scheduling preferences, patient physiological constraints (e.g., regarding IMDs, pregnancy, patient demographics (e.g., height, weight, body size, age, and other important parameters for determining the appropriate imaging device and acquisition protocols/parameters). The imaging order may be electronically entered into the RIS 134 which facilitates scheduling the exam for the patient with a medical imaging device capable of fulfilling the order (e.g., based on the type of the exam ordered) at a medical imaging site of amongst the medical imaging sites 136_{1-N}.

Prior to performance of the exam, the recommended acquisition protocol (i.e., the planned acquisition protocol) is determined for the exam based on the order information (e.g., the modality, the anatomical ROI, the clinical indication, patient parameters, and other factors). The acquisition protocol accounts for not only the modality, but the technical acquisition parameters applicable to the modality that control the resulting image quality, resolution, radiation dose exposure to the patient, and scan time. For example, the acquisition parameters can include various imaging parameters, such as the field of view, slice thickness (as applied to CT and MRI), resolution, plane of acquisition, and matrix size (as applied to CT and MRI). The acquisition parameters can also include timing and sequency parameters (e.g., scan timing, sequency type (for MRI, such as T1-weighted, T2-weighted, etc.), gating or synchronization. The acquisition parameters can also include dose and exposure settings (for radiation-based modalities such as CT, X-ray, and nuclear medicine), including radiation dose, tube current and voltage settings, exposure time, and dose reduction parameters.

Acquisition protocols vary significantly between different types of medical imaging exams due to the unique principles, purposes, and technological characteristics of each modality. Acquisition protocols further vary for different types of imaging exams within the same modality because the clinical objectives, anatomy being examined, patient factors, and technical requirements can differ significantly. These variations ensure that the imaging captures the necessary information with optimal quality, safety, and efficiency. Generally, the recommended or planned acquisition protocol is tailored to provide a diagnostically acceptable level of image quality while minimizing radiation dose exposure (for radiation-based modalities such as CT, X-ray, and nuclear medicine), and minimizing scan time.

The planned acquisition protocol for an imaging exam may correspond to a predefined, standard acquisition protocol determined for the modality, the ROI, the clinical indication and possibly patient factors. For example, many clinical guidelines and/or medical imaging system providers provide pre-configured standardized protocols tailored to specific imaging systems and clinical scenarios. Additionally, or alternatively, the planned acquisition protocol may correspond to a manually defined protocol (e.g., provided by a radiologist) and/or an automatically generated protocol generated via intelligent protocol planning optimization software. For example, in many clinical scenarios, a radiologist reviews the order information and determines the recommended protocol based on the modality, the clinical question, the ROI, patient-specific factors (e.g., allergies, kidney function, prior imaging results) and institutional guidelines and best practices. In some implementations, the radiologist may tailor a standardized protocol defined for a particular modality, ROI, clinical indication and in some implementation's patient factors (e.g., age, gender, patient size, etc.) for a particular patient and order, or leave the standardized protocol unmodified.

In other implementations, intelligent protocol management software can automatically determine the recommend or planned protocol based on the order information. Software that automatically generates the optimal acquisition protocol for a medical imaging exam typically leverages advanced algorithms, clinical guidelines, patient data, and imaging system capabilities. These tools aim to standardize and optimize imaging protocols to ensure diagnostic accuracy, efficiency, and patient safety, often leveraging AI. For example, intelligent protocol management software is provided by various medical imaging device providers that automate protocol selection based on patient characteristics and clinical indications. These software systems use AI and machine learning to guide technologists in selecting optimal imaging parameters based on the clinical context, employ pre-loaded and customizable protocols, and provide for automatic parameter adjustments based on patient size and clinical indication.

In various embodiments, the protocol planning component 108 can provide for setting or defining the planned protocols to be applied for medical imaging exams to be performed via the respective medical imaging sites 136_{1-N}. In this regard, based on reception of a new order for a medical imaging exam at the RIS 134, the protocol planning component 108 can generate (e.g., automatically) or facilitate generating (e.g., by a radiologist or the like) the planned protocol for the medical imaging exam based on the order information and relevant patient factors. For example, in some embodiments, the protocol panning component 108 can correspond to intelligent protocol planning software that automatically selects a pre-configured, standard protocol for the exam (based on the order information) and/or that automatically customizes parameters of the standard protocol for the clinical indication and patient size, age, gender, etc. In other embodiments, the protocol planning component 108 can include or correspond to an interactive protocol planning application that facilitates receiving user input (e.g., radiologist input or the like) selecting, defining and/or customizing the planned protocol. Information defining the planned (i.e., recommended) protocol to be employed for new imaging exam orders arriving at the RIS 134 can be stored in memory 128 (e.g., as planned protocol data 120) and/or the RIS 134.

In association with performing an imaging exam ordered for a patient, the planned protocol for the exam is provided, sent or otherwise communicated to (e.g., by protocol management system 102 via communication component 106 and/or the RIS 134) the imaging site (of amongst medical imaging sites 136_{1-N}) and/or the actual imaging device used to perform the exam. The technician operating the imaging device is then directed to perform the exam using the planned protocol. The exam is then performed and the actual protocol used for the exam (i.e., the performed protocol) is realized. The performed protocol may be recorded with the medical imaging scan data acquired via the exam and sent to the RIS 134 and/or back to the protocol management system 102. For example, information defining the performed protocol data for respective exams performed via the respective medical imaging sites 136_{1-N} can be stored in memory 128 (e.g., as performed protocol data 122), at the RIS 134, or another accessible system or device.

As noted in the Background section, the performed protocol used for the exam may vary from the planned protocol for a variety of reasons. However, because different medical imaging devices and/or sites (e.g., of amongst medical imaging sites 136_{1-N}) often employ different data formats (e.g., having different terminologies and data structures) to record the performed protocols relative to that used for the planned protocols, existing protocol management system cannot efficiently compare standardized protocols with actual performed protocols in association with monitoring and assessing adherence to the planned protocols.

To solve this problem, protocol management system 102 combines a rules-based technique with an AI technique to enable automatically mapping and comparing planned imaging protocols with their corresponding performed imaging protocols, wherein the planned imaging protocols are provided in a first data format and the performed imaging protocols are provided in one or more second data formats. In this regard, the planned imaging protocols can include or correspond to planned imaging protocols for respective imaging exams performed via the medical imaging sites 136_{1-N} (e.g., for orders entered at the RIS 134 or the like), and the performed imaging protocols can include the corresponding actual protocols used for the performance of the respective imaging exams via the medical imaging sites 136_{1-N}. As noted above, the information (i.e., data files) recording the planned protocols for the respective exams is represented in system 100 as planned protocol data 120, and the information recording the performed protocols for the respective exams is represented in system 100 as performed protocol data 122.

In various embodiments, the planned protocol data 120 is provided in a first data format, and the performed protocol data 122 is provided in one or more second data formats different from the first data format. The first data format and the one or more second data formats can vary. For example, in some implementations, the first data format comprises the JavaScript Object Notation (JSON) format, and the one or more second data formats comprise the Comma-Separated Values (CSV) format, however, the first data format and the second data format are not restricted to JSON and CVS respectively, so long as the first and second data formats are different. JSON is a lightweight data-interchange format that is easy for humans to read and write and easy for machines to parse and generate. It represents data in a structured format using key-value pairs and hierarchical structures. On the other hand, CSV is a plain-text format that uses commas (or other delimiters like tabs or semicolons) to separate values in a table-like structure. Each row represents a record, and each column represents a field.

The rule-based technique involves performing a similarity mapping process (e.g., by similarity mapping component 110) between pairs of medical imaging protocol data, wherein the pairs respectively comprise planned imaging protocol data for medical imaging exams in a first format and performed imaging protocol data for the medical imaging exams in one or more second data formats. The output of the similarity mapping process comprises, for each pair, generating a similarity matrix that represents measures of similarity of respective acquisition protocol parameters between the planned and performed imaging protocol.

The AI technique involves training (e.g., via training component 112) a neural network model 114 to estimate similarity scores for an initial set of training data pairs using similarity matrices generated by the similarity mapping component 110 for the training pairs as input to the neural network model. In one or more embodiments, the training comprises training the neural network model using a supervised machine learning process and ground truth information for respective ones of the training data pairs identifying ground truth similarity scores. Once trained, the inferencing component 116 employs the trained version of the neural network model 114 to estimate similarity scores for new pairs of medical imaging protocol data based on new similarity matrices generated for the new pairs by the similarity mapping component 110, wherein the new pairs respectively comprise new planned imaging protocol data in the first data format (e.g., included in the planned protocol data 120) and new performed imaging protocol data a second data format (e.g. included in the performed protocol data 122). The similarity scores output by the trained version of the neural network model 114 indicate how similar (or deviant) respective performed protocols used for actual imagining exams are relative to their corresponding planned protocols.

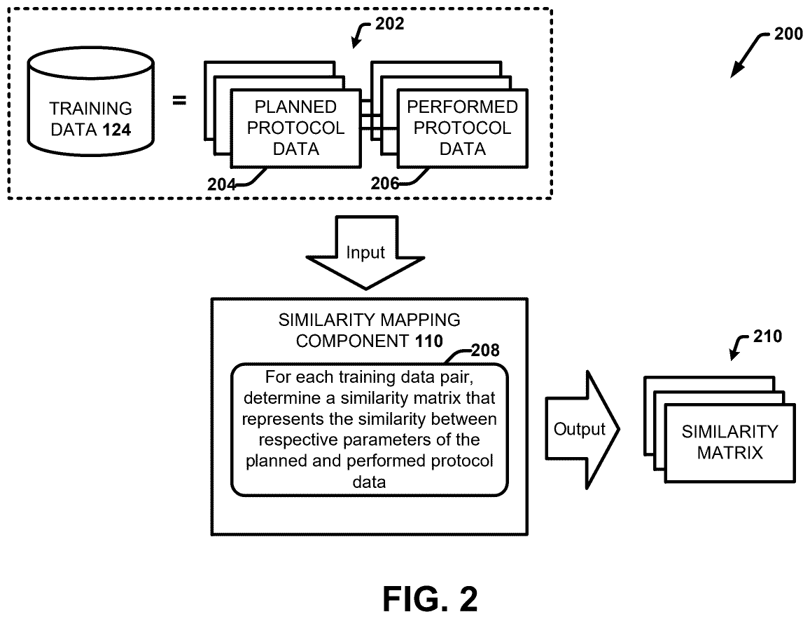

FIG. 2 illustrates a high-level similarity mapping process 200 in accordance with one or more embodiments described herein. With reference to FIGS. 1 and 2, process 200 corresponds to an initial similarity mapping process performed by the similarity mapping component 110 using training data 124. In this regard, the training data 124 can comprise a plurality of pairs 202 of medical imaging protocol data. The number of pairs 202 can vary and include any suitable number of training data samples (e.g., tens, hundreds, thousands, etc.). Each pair of medical imaging protocol data represents a medical imaging exam performed on a patient and includes planned protocol data 204 that defines the planned protocol for the exam, and performed protocol data 206 that defines the performed protocol used for the exam. In some embodiments, the pairs 202 can represent a variety of different types of medical imaging exams, wherein the different types vary with respect to modality, ROI, clinical indication, patient factors (e.g., age, gender, size, etc.). The planned and performed acquisition protocols for the respective pairs can also vary.

In accordance with the disclosed techniques, the planned protocol data 204 for each pair is provided in a first data format, and the performed protocol data 206 for each pair is provided in one or more second formats different from the first data format. The first data format and the one or more second data formats can vary so long as the first data format and the one or more second data formats are different. In various embodiments, the first and second data formats can respectively include different text-based data formats such as JSON, CVS, and extensible Markup Language (XML). These and other text-based formats store data as plain text, often human-readable in addition to being machine-readable. The different text-based formats vary with respect to the structure and terminology used to define respective parameters of the acquisition protocols.

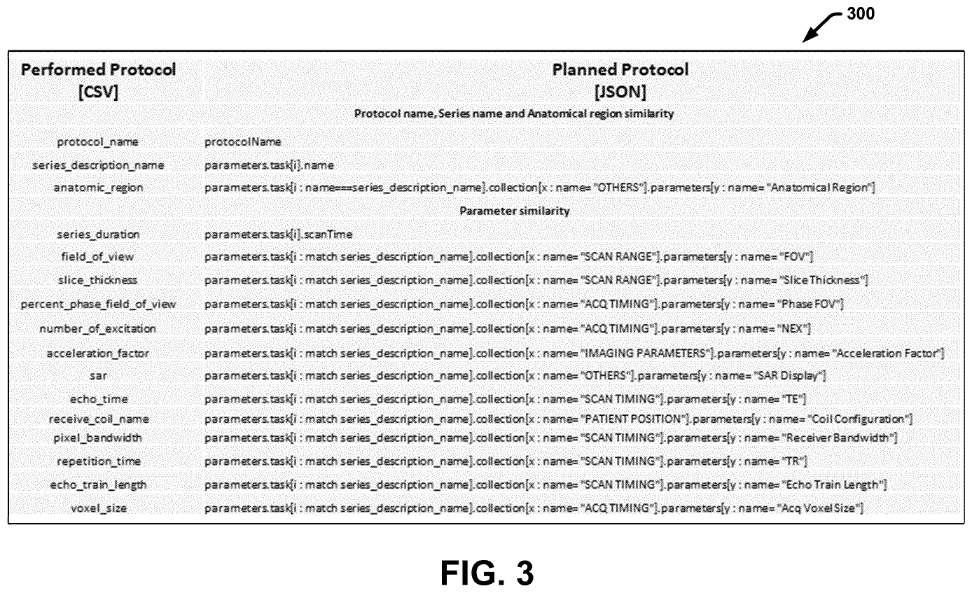

For example, FIG. 3 illustrates a table 300 comprising an example planned protocol in a first data format and a corresponding performed protocol in a second data format, in accordance with one or more embodiments described herein. In this example, the first data format of the planned protocol comprises JSON and the second data format of the performed protocol comprises CSV. Table 300 aligns respective parameters of the performed protocol in CVS with corresponding parameters of the planned protocol in JSON. In this example, the imaging exam represented corresponds to an MRI exam and the parameters correspond to those applicable to an MRI exam. For example, the parameters include protocol name, series description name, anatomic region, series duration, field of view, slice thickness, percent phase field of view, number of excitations, acceleration factor, specific absorption rate, echo time, receive coil name, pixel bandwidth, repetition time, echo train length and voxel size. It should be appreciated that different parameters are applicable to different types of medical imaging exams with respect to modality and other factors. As can be seen in Table 300, the terms and structure of the parameters in the CSV format are different from the corresponding parameters in the JSON format.

With reference back to FIG. 2 in view of FIGS. 1 and 3, in accordance with the initial similarity mapping process 200, at 208, for each training data pair of the pairs 202, the similarity mapping component 110 determines a similarity matrix or a similarity vector that represents the similarity between respective parameters of the planned protocol data 204 and the performed protocol data 206. In other words, for each pair, at 208 the similarity mapping component 110 determining measures of similarity between respective parameters of a defined set of parameters included in the planned protocol data 204 and the performed protocol data 206 and generates a similarity matrix for the pair comprising the measures of similarity.

For example, the similarity mapping component 110 can employ predefined mapping information (e.g., stored in memory 128 or another accessible memory) that maps respective parameters in the first data format to corresponding parameters in the second data format. Using the predefined mapping information, for each training data pair, the similarity mapping component 110 maps respective parameters of the planned protocol data 204 in the first data format to corresponding parameters of the performed protocol data 206 in the second data format. For each parameter, the similarity mapping component 110 determines a measure of similarity between the planned and performed parameters based on whether and to what degree the property values (e.g., actual terms and/or values) are the same. For example, the measure of similarity for each parameter can comprise a value between 0.0 and 1.0, wherein a value of 0.0 corresponds to the lowest degree of similarity, wherein a value of 1.0 corresponds to the highest degree of similarity, and wherein the value can range between 0.0 and 1.0. The similarity matrix or similarity vector generated for each pair comprises the collection of similarity measures, one similarity measure for each parameter. The size of the similarity matrix or similarity matrix vector is thus based on the number of parameters included in the planned protocol data 204. For example, as applied to an MRI exam having planned protocol data corresponding to that illustrated in table 300, the number of parameters evaluated and thus represented in the similarity matrix is 16 total parameters (e.g., protocol name, series description name, anatomic region, series duration, field of view, slice thickness, percent phase field of view, number of excitations, acceleration factor, specific absorption rate, echo time, receive coil name, pixel bandwidth, repetition time, echo train length and voxel size). The output of the initial similarity mapping process 200 is a plurality of similarity matrices 210, one matrix for each training data pair.

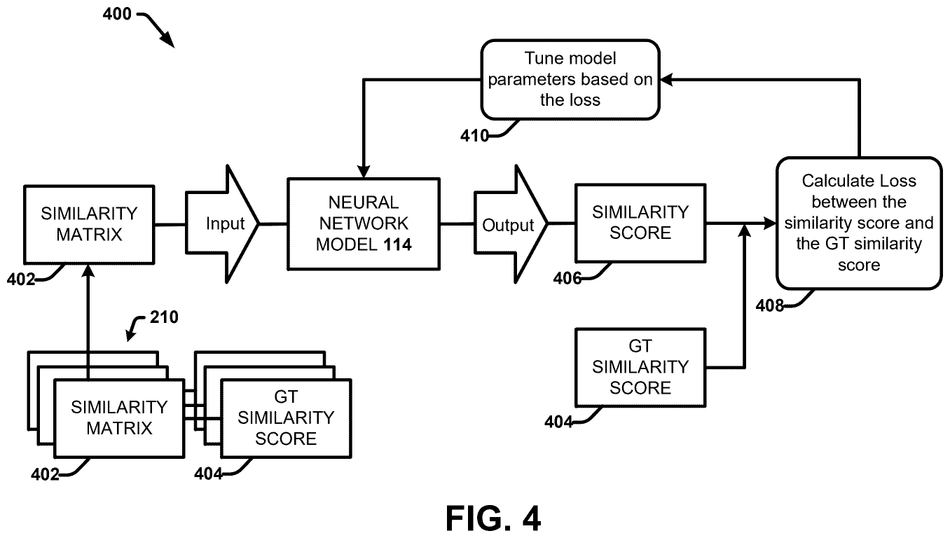

FIG. 4 illustrates an example training process 400 for training neural network model 114 to estimate similarity scores, in accordance with one or more embodiments described herein. With reference to FIGS. 1-4, training process 400 corresponds to an example training process performed by training component 112. Training process 400 involves training neural network model 114 to estimate similarity scores respectively represented by the respective similarity matrices 210 generated for the training data pairs 202 via the initial similarity mapping process 200. In this regard, the neural network model 114 can comprise a feedforward neural network (FNN) model configured to process a similarity matrix 402 as input and generate a similarity score 406 as output. The number of layers of the neural network model 114, the types of the layers, and the number of parameters in each layer can vary.

Training process 400 is a supervised machine learning process, wherein each similarity matrix 402 of the respective similarity matrices 210 is paired with a ground truth (GT) similarity score 404. The GT similarity scores can be manually defined. In accordance with conventional supervised machine learning, training process 400 involves feeding a similarity matrix 402 as input to the neural network model 114 which generates a similarity score 406 as output. At 408, the training component 112 calculates a loss metric value between the similarity score 406 and the GT similarity score 404 using a suitable loss function (which can vary), such as mean squared error, Dice, entropy loss or the like. At 410, the training component 112 then tunes the parameters of the neural network model 114 based on the loss (e.g., adjusting model parameter weights and biases based on the loss function's gradients). Training component 112 repeats this process for each similarity matrix included in the set of similarity matrices 210 (or until convergence is reached or another suitable training conclusion event occurs). The result of training process 400 is the generation of a trained version of the neural network model 114 configured to receive a similarity matrix corresponding to similarity matrix 402 as input and generate a similarity score corresponding to similarity score 406, wherein the similarity score represents a measure of similarity between planned and performed protocol data for a medical imaging exam. For example, the similarity score 406 can correspond to a percentage score, wherein the higher the percentage score, the greater the degree of similarity.

In this manner, as opposed to training the neural network model 114 to assess similarity between raw text of the first and second protocol data formats, the neural network model assesses similarity based on a similarity matrix that represents the semantic relationships between the respective planned and performed protocols.

Once trained, the protocol management system 102 can employ the trained version of the neural network model 114 to estimate similarity scores for new pairs of medical imaging protocol data based on new similarity matrices generated for the new pairs using the similarity mapping process 200, wherein the new pairs respectively comprise new planned imaging protocol data in the first data format and new performed imaging protocol data the one or more second data formats.

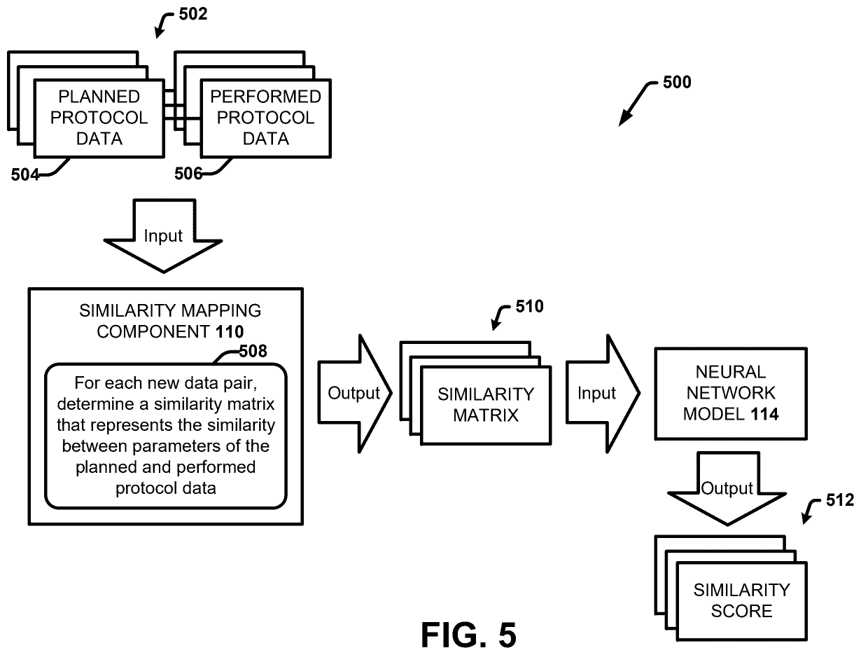

For example, FIG. 5 illustrates an example runtime process 500 for generating similarity scores for new pairs of planned and performed protocol data, in accordance with one or more embodiments described herein. With reference to FIG. 5 in view of FIGS. 1-4, process 500 corresponds to an example process the can be performed by the protocol management system 102 following generation of a trained version of the neural network model 114. Similar to process 200, process 500 involves performing a similarity mapping process by the similarity mapping component 110 to generate similarity matrices 510 for respective pairs 502 of medical imaging protocol data. The number of pairs 202 can vary. Each pair of medical imaging protocol data of amongst pairs 502 represents a medical imaging exam performed on a patient and includes planned protocol data 504 (included in planned protocol data 120) that defines the planned protocol for the exam and performed protocol data 506 (included in performed protocol data 122) that defines the performed protocol used for the exam. The pairs 502 can represent a variety of different types of medical imaging exams, wherein the different types vary with respect to modality, ROI, clinical indication, patient factors (e.g., age, gender, size, etc.). The planned and performed acquisition protocols for the respective pairs 502 can also vary.

In accordance with the disclosed techniques, the planned protocol data 504 for each pair is provided in the first data format, and the performed protocol data 506 for each pair is provided in the one or more second formats different from the first data format. That is the first data format can correspond to the same first data format used for the planned protocol data 204 and the one or more second data formats can correspond to the same one or more second data formats of the performed protocol data 206. For example, in some embodiments, the first data format can comprise JSON and the one or more second data formats can comprise CSV.

In accordance with process 500, at 508 for each new data pair of the pairs 502, the similarity mapping component 110 determines a similarity matrix or a similarity vector that represents the similarity between respective parameters of the planned protocol data 504 and the performed protocol data 506. In other words, for each pair, at 508 the similarity mapping component 110 determining measures of similarity between respective parameters of a defined set of parameters included in the planned protocol data 504 and the performed protocol data 506 and generates a similarity matrix for the pair comprising the measures of similarity.

For example, the similarity mapping component 110 can employ predefined mapping information (e.g., stored in memory 128 or another accessible memory) that maps respective parameters in the first data format to corresponding parameters in the second data format. Using the predefined mapping information, for each new data pair, the similarity mapping component 110 maps respective parameters of the planned protocol data 504 in the first data format to corresponding parameters of the performed protocol data 506 in the second data format. For each parameter, the similarity mapping component 110 determines a measure of similarity between the planned and performed parameters based on whether and to what degree the property values (e.g., actual terms and/or values) are the same. For example, the measure of similarity for each parameter can comprise a value between 0.0 and 1.0, wherein a value of 0.0 corresponds to the lowest degree of similarity, wherein a value of 1.0 corresponds to the highest degree of similarity, and wherein the value can range between 0.0 and 1.0. The similarity matrix or similarity vector generated for each pair comprises the collection of similarity measures, one similarity measure for each parameter. The size of the similarity matrix or similarity matrix vector is thus based on the number of parameters included in the planned protocol data 504.

The output of the similarity mapping component 110 for each pair is similarity matrix. Process 500 is described in association with the similarity mapping component 110 processing a plurality of pairs 502 and thus similarity matrices 510 correspond to the respective similarity matrices generated for each pair by the similarity mapping component 110. It should be appreciated however that the protocol management system 102 can process individual pairs of new planned and performed protocol data for new medical imaging exams as they are obtained (e.g., in response to performance of the exam and reception of the actual performed protocol data for the exam at the RIS 134 and/or the protocol management system 102).

Continuing with process 500, once generated, the inferencing component 116 supplies the respective similarity matrices as input to the trained version of the neural network model 114 (e.g., individually), which generates corresponding similarity scores 512 for the respective matrices 510 as output (e.g., one similarity score per similarity matrix.

To this end, the similarity scores 512 output by the trained version of the neural network model 114 indicate how similar (or deviant) respective performed protocols used for actual imagining exams are relative to their corresponding planned protocols. In various embodiments, these similarity scores can be used to automatically identify imaging exams and corresponding imaging systems (and/or imaging devices, technicians, etc.) that deviate from the recommended protocols, enabling efficient remediation. The disclosed techniques can further enable root cause analysis as to the basis why and its impact on resulting image quality, radiation dose exposure, and other performance valuation metrics.

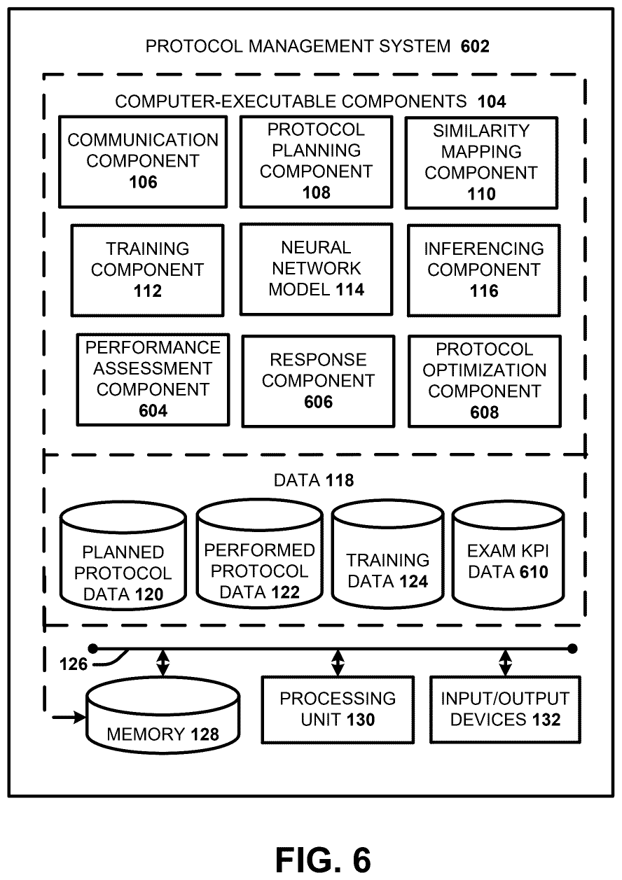

For example, FIG. 6 illustrates another example protocol management system 602 in accordance with one or more embodiments described herein. With reference to FIG. 6 in view of FIGS. 1-5, protocol management system 602 corresponds to protocol management system 102 with the addition of performance assessment component 604, response component 606, protocol optimization component 608, and exam KPI (key performance indicator) data 610. Repetitive description of like elements employed in respective embodiments is omitted for sake of brevity.

The performance assessment component 604 can perform analytical evaluation of the similarity scores 512 to automatically identify imaging exams and corresponding imaging systems (and/or imaging devices, technicians, etc.) that deviate from the recommended protocols, enabling efficient remediation. Likewise, the performance assessment component 604 can perform analytical evaluation of the similarity scores 512 to automatically identify imaging exams and corresponding imaging systems (and/or imaging devices, technicians, etc.) that adhere to the recommended protocols. The response component 606 can further provide for performing various responses based on identification of imaging exams and corresponding imaging systems (and/or imaging devices, technicians, etc.) that deviate from and/or adhere to the recommended protocols.

For example, the performance assessment component 604 can analyze trends in protocol adherence across departments or facilities and identify recurring deviations to address systemic issues. In some implementations, the response component 606 can be configured to flag performed imaging protocols for review by radiologists with similarity scores below a threshold similarity score. The response component 604 can also provide feedback to technologists or radiologists on errors or deviations to guide future scans. The response component 606 can further generate detailed report information comparing performed scans to standard protocols. For example, the detailed report information can identify the performed protocol, the imaging site and/or imaging device that performed the corresponding imaging exam using the performed protocol, the planned protocol for the exam, and the resulting similarity score. The response component 606 can also store the report information as protocol compliance information with the performed imaging scan data for audits and future analysis. In some cases, the response component 606 can be configured to notify the imaging site and/or the imaging device regarding the similarity score being below the threshold. Additionally, or alternatively, the response component 606 can be configured to monitor and track additional imaging exams of that type performed at the medical imaging site based on detection of usage of the performed protocol that deviates from the planned protocol using heightened surveillance.

In some embodiments, in association with evaluating the similarity scores 512, the performance assessment component 604 can also analyze performance metrics (KPIs) relevant to the imaging exams. In this regard, the recommended or planned imaging protocols are generally tailored to achieve medical images having a requisite level of image quality acceptable for diagnostic purposes and minimizing radiation dose and scan time. In various embodiments, the exam KPI data 610 can include reference performance metric values that reflect the expected (and acceptable) image quality measures (e.g., a signal-to-noise metric (SNR), a contrast resolution metric, a spatial resolution metric, and artifact measure, etc.) for images resulting from scans performed using the respective planned protocols. The exam KPI data 610 can also include reference performance metric values that reflect the expected (and acceptable) radiation doses resulting from scans performed using the respective planned protocols. The exam KPI data 610 can also include corresponding image quality metrics and dose metrics determined for the performed exams in accordance with the performed protocols.

In some embodiments, the performance assessment component 604 can identify a subset of the new pairs with similarity scores below a threshold similarity score, and determine for respective pairs of the subset, whether one or more performance metrics (e.g., image quality metrics, dose metrics, image scan time metrics) for imaging scans of the subset satisfy one or more acceptability criterion (e.g., acceptable values for the corresponding performance metrics). With these embodiments, the response component 606 can be configured to perform a response such as generating a notification, flagging the performed protocol, performing heightened monitoring, or the like, based on a determination that the performance metrics for a scan that did not follow the recommended protocol, do not satisfy the acceptability criterion. Likewise, the performance assessment component 604 can identify a subset of the new pairs with similarity scores above a threshold similarity score, and determine for respective pairs of the subset, whether one or more performance metrics for imaging scans of the subset satisfy one or more acceptability criterion. With these embodiments, the response component 606 can be configured to perform a positive response such as generating a notification and providing a reward to entities (e.g., imaging sites and/or imaging devices) that adhere to the recommended protocols.

In some embodiments, the protocol optimization component 608 can also use performed protocols that differ from their corresponding recommended protocols yet result in improvements to the corresponding reference performance metrics. For example, the performance assessment component 604 can identify a subset of the new pairs 502 with similarity scores below a threshold similarity score. The performance assessment component 604 can further determine, for respective pairs of the subset, differences between one or more reference performance metrics for the planned protocols of the subset and actual performance metrics for the performed imaging scans of the subset that adhere to the new performed imaging protocol data. The performance assessment component 604 can further identify one or more pairs of the subset associated with differences that correspond to an improvement to the one or more performance metrics (e.g., an increase in image quality, a decrease in radiation dose, and/or a decrease in scan time). The protocol optimization component 608 can further determine a modification to the corresponding planned imaging protocol data that facilitates the improvement, resulting in optimized standard protocol data for a particular exam type (e.g., based on modality, ROI, clinical indication and optionally patient factors). The protocol optimization component 608 can further control usage of the optimized standard protocol data as the planned protocol data for new order for exams of that type (e.g., by the protocol planning component 108).

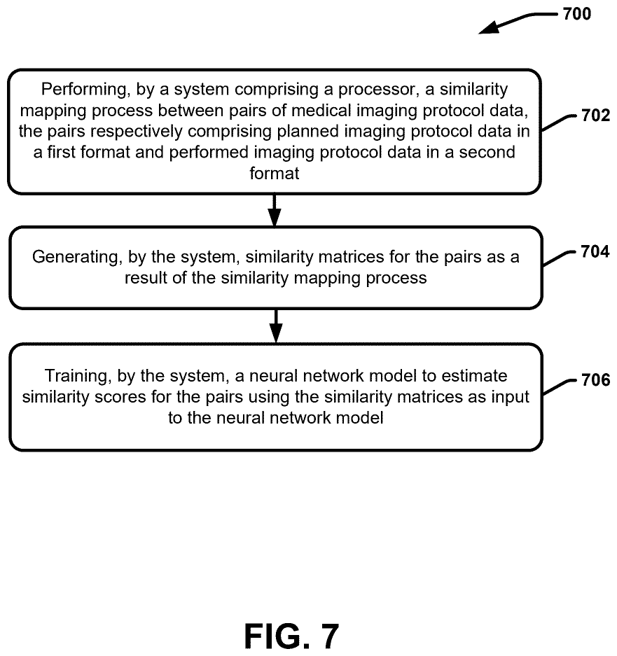

FIG. 7 illustrates an example computer-implemented method 700 for medical imaging protocol mapping using AI, in accordance with one or more embodiments described herein. With reference to FIG. 7 in view of FIGS. 1-6, method 700 comprises, at 702 performing, by a system comprising a processor (e.g., protocol management system 102, protocol management system 602, or the like), a similarity mapping process (e.g., process 200) between pairs of medical imaging protocol data (e.g., via similarity mapping component 110, the pairs respectively comprising planned imaging protocol data for a medical imaging exam in a first format and performed imaging protocol data for the medical imaging exam in a second format. In this regard, the pairs can correspond to training pairs 202. At 704, method 700 comprises generating, by the system, similarity matrices for the pairs as a result of the similarity mapping process (e.g., similarity matrices 210). At 706, method 700 comprises training, by the system (e.g., via training component 112), a neural network model (e.g., neural network model 114) to estimate similarity scores for the pairs using the similarity matrices as input to the neural network model, as described with reference to FIG. 4 and training process 400.

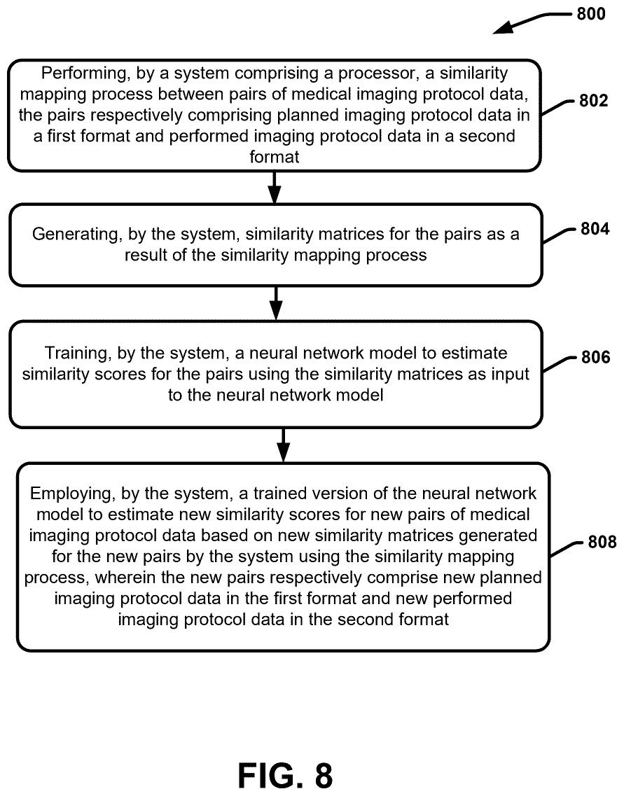

FIG. 8 illustrates another example computer-implemented method 800 for medical imaging protocol mapping using AI, in accordance with one or more embodiments described herein. With reference to FIG. 8 in view of FIGS. 1-6, method 800 comprises, at 802 performing, by a system comprising a processor (e.g., protocol management system 102, protocol management system 602, or the like), a similarity mapping process (e.g., process 200) between pairs of medical imaging protocol data (e.g., via similarity mapping component 110, the pairs respectively comprising planned imaging protocol data for a medical imaging exam in a first format and performed imaging protocol data for the medical imaging exam in a second format. In this regard, the pairs can correspond to training pairs 202. At 804, method 700 comprises generating, by the system, similarity matrices for the pairs as a result of the similarity mapping process (e.g., similarity matrices 210). At 806, method 700 comprises training, by the system (e.g., via training component 112), a neural network model (e.g., neural network model 114) to estimate similarity scores for the pairs using the similarity matrices as input to the neural network model. At 808, method 800 comprises employing, by the system, a trained version of the neural network model to estimate new similarity scores for new pairs of medical imaging protocol data based on new similarity matrices generated for the new pairs by the system using the similarity mapping process (e.g., corresponding to process 200), wherein the new pairs respectively comprise new planned imaging protocol data in the first format and new performed imaging protocol data in the second format. For example, the new pairs can correspond to pairs 502 and the new similarity scores can correspond to similarity scores 512.

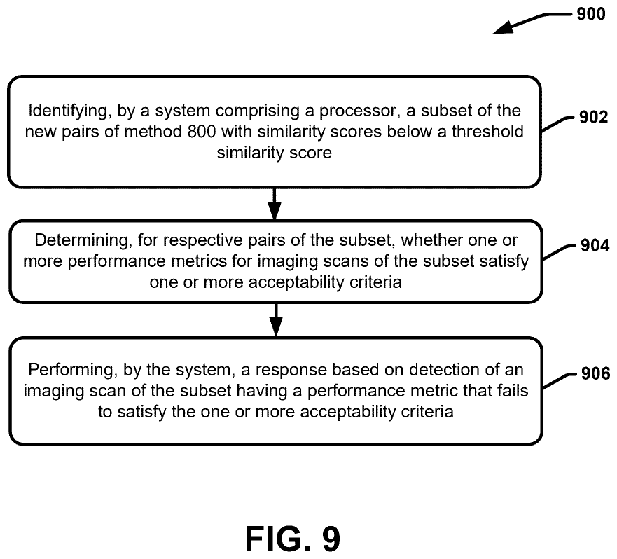

FIG. 9 illustrates an example computer-implemented method 900 for monitoring adherence to standardized medical protocols, in accordance with one or more embodiments described herein. Method 900 corresponds to an example method involving performing analytical evaluation of the new similarity scores generated via method 800. With reference to FIG. 9 in view of FIGS. 1-8, at 902, method 900 comprises, identifying, by a system comprising a processor (e.g., protocol management system 602), a subset of the new pairs of method 800 with similarity scores below a threshold similarity score (e.g., via performance assessment component 604). At 904, method 900 comprise determining, for respective pairs of the subset, whether one or more performance metrics for imaging scans of the subset satisfy one or more acceptability criteria. For example, the one or more performance metrics can include a quality metric that represents a measure of image quality of the imaging scans and the one or more acceptability criteria can include an acceptable value for the quality metric. In another example, as applied to imaging scans that uses radiation (e.g., X-ray, CT, and nuclear medicine), the one or more performance metrics can include a dose metric that represents an amount of radiation dose exposed to the patient during the scan, and the one or more acceptability criteria can include an acceptable value for the dose metric. At 906, method 900 comprises performing, by the system, a response based on detection of an imaging scan of the subset having a performance metric that fails to satisfy the one or more acceptability criteria (e.g., via response component 606). For example, the response can include any of the responses described with reference to response component 606 (e.g., generating a report, generating a notification, performing heightened monitoring, flagging the performed protocol, etc.).

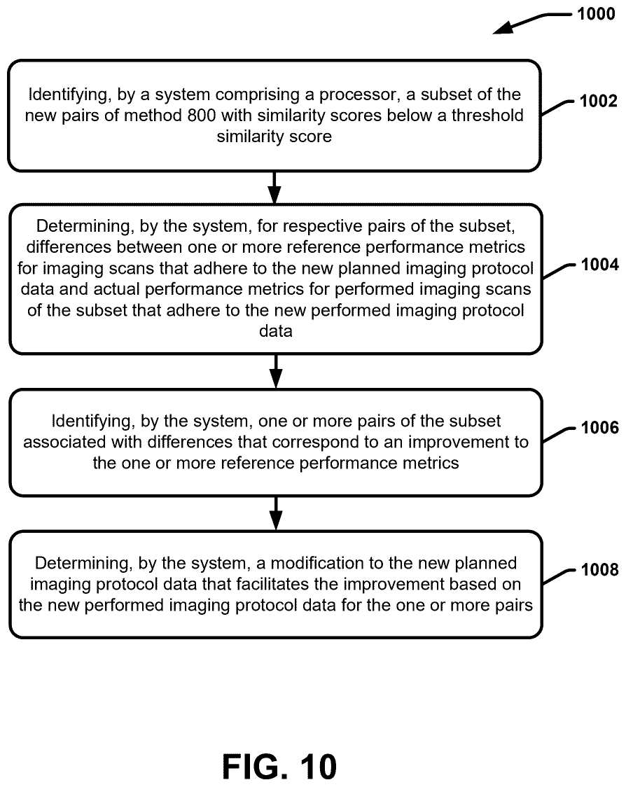

FIG. 10 illustrates another example computer-implemented method 1000 for monitoring adherence to standardized medical protocols, in accordance with one or more embodiments described herein. Method 10000 corresponds to another example method involving performing analytical evaluation of the new similarity scores generated via method 800. With reference to FIG. 10 in view of FIGS. 1-9, at 1002, method 1000 comprises, identifying, by a system comprising a processor (e.g., protocol management system 602), a subset of the new pairs of method 800 with similarity scores below a threshold similarity score (e.g., via performance assessment component 604). At 1004, method 1000 comprise determining, for respective pairs of the subset, differences between one or more reference performance metrics for imaging scans that adhere to the new planned imaging protocol data and actual performance metrics for performed imaging scans of the subset that adhere to the new performed imaging protocol data (e.g., via performance assessment component 604). For example, the reference performance metrics and the actual performance metrics can include one or more image quality metrics (e.g., an SNR value, a measure artifacts, a temporal resolution value, etc.) and/or one more radiation dose metrics. At 1006, method 100 comprises identifying, by the system, one or more pairs of the subset associated with differences that correspond to an improvement to the one or more reference performance metrics. For example, the reference performance metrics can provide the expected (and/or requisite) image quality metric values and/or dose metrics to be realized via the planned protocol. To this end, at 1006, the performance assessment component 604 can identify performed protocols with performance metrics (e.g., image quality, radiation dose, etc.) that are better (e.g., better image quality, lower radiation dose, etc.) than the reference metrics. At 1008, method 100 comprises determining, by the system, a modification to the new planned imaging protocol data that facilitates the improvement based on the new performed imaging protocol data for the one or more pairs. In other words, the performance assessment component 604 can determine what parameter differences account for the improvement and adjust the recommended or standardized protocol for the corresponding type of imaging exam (e.g., based on modality, ROI, clinical indication and patient factors) as included in reference guideline information used by the protocol planning component 108 accordingly

In order to provide a context for the various aspects of the disclosed subject matter, FIGS. 11 and 12 as well as the following discussion are intended to provide a brief, general description of a suitable environment in which the various aspects of the disclosed subject matter may be implemented.

With reference to FIG. 11, a suitable environment 1100 for implementing various aspects of this disclosure includes a computer 1112. The computer 1112 includes a processing unit 1114, a system memory 1116, and a system bus 1118. The system bus 1118 couples system components including, but not limited to, the system memory 1116 to the processing unit 1114. The processing unit 1114 can be any of various available processors. Dual microprocessors and other multiprocessor architectures also can be employed as the processing unit 1114.

The system bus 1118 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1116 includes volatile memory 1120 and nonvolatile memory 1122. The basic input/output system (BIOS), containing the basic routines to transfer information between elements within the computer 1112, such as during start-up, is stored in nonvolatile memory 1122. By way of illustration, and not limitation, nonvolatile memory 1122 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 1120 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 1112 also includes removable/non-removable, volatile/non-volatile computer storage media. FIG. 11 illustrates, for example, a disk storage 1124. Disk storage 1124 includes, but is not limited to, devices like a magnetic disk drive, floppy disk drive, tape drive, Jaz drive, Zip drive, LS-110 drive, flash memory card, or memory stick. The disk storage 1124 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1124 to the system bus 1118, a removable or non-removable interface is typically used, such as interface 1126.

FIG. 11 also depicts software that acts as an intermediary between users and the basic computer resources described in the suitable operating environment 1100. Such software includes, for example, an operating system 1128. Operating system 1128, which can be stored on disk storage 1124, acts to control and allocate resources of the computer system 1112. System applications 1130 take advantage of the management of resources by operating system 1128 through program modules 1132 and program data 1134, e.g., stored either in system memory 1116 or on disk storage 1124. It is to be appreciated that this disclosure can be implemented with various operating systems or combinations of operating systems.

A user enters commands or information into the computer 1112 through input device(s) 1136. Input devices 1136 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1114 through the system bus 1118 via interface port(s) 1138. Interface port(s) 1138 include, for example, a serial port, a parallel port, a game port, and a universal serial bus (USB). Output device(s) 1140 use some of the same type of ports as input device(s) 1136. Thus, for example, a USB port may be used to provide input to computer 1112, and to output information from computer 1112 to an output device 1140. Output adapter 1142 is provided to illustrate that there are some output devices 1140 like monitors, speakers, and printers, among other output devices 1140, which require special adapters. The output adapters 1142 include, by way of illustration and not limitation, video and sound cards that provide a means of connection between the output device 1140 and the system bus 1118. It should be noted that other devices and/or systems of devices provide both input and output capabilities such as remote computer(s) 1144.

Computer 1112 can operate in a networked environment using logical connections to one or more remote computers, such as remote computer(s) 1144. The remote computer(s) 1144 can be a personal computer, a server, a router, a network PC, a workstation, a microprocessor based appliance, a peer device or other common network node and the like, and typically includes many or all of the elements described relative to computer 1112. For purposes of brevity, only a memory storage device 1146 is illustrated with remote computer(s) 1144. Remote computer(s) 1144 is logically connected to computer 1112 through a network interface 1148 and then physically connected via communication connection 1150. Network interface 1148 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1150 refers to the hardware/software employed to connect the network interface 1148 to the bus 1118. While communication connection 1150 is shown for illustrative clarity inside computer 1112, it can also be external to computer 1112. The hardware/software necessary for connection to the network interface 1148 includes, for exemplary purposes only, internal and external technologies such as, modems including regular telephone grade modems, cable modems and DSL modems, ISDN adapters, and Ethernet cards.

FIG. 12 is a schematic block diagram of a sample-computing environment 1200 with which the subject matter of this disclosure can interact. The system 1200 includes one or more client(s) 1210. The client(s) 1210 can be hardware and/or software (e.g., threads, processes, computing devices). The system 1200 also includes one or more server(s) 1230. Thus, system 1200 can correspond to a two-tier client server model or a multi-tier model (e.g., client, middle tier server, data server), amongst other models. The server(s) 1230 can also be hardware and/or software (e.g., threads, processes, computing devices). The servers 1230 can house threads to perform transformations by employing this disclosure, for example. One possible communication between a client 1210 and a server 1230 may be in the form of a data packet transmitted between two or more computer processes.

The system 1200 includes a communication framework 1250 that can be employed to facilitate communications between the client(s) 1210 and the server(s) 1230. The client(s) 1210 are operatively connected to one or more client data store(s) 1220 that can be employed to store information local to the client(s) 1210. Similarly, the server(s) 1230 are operatively connected to one or more server data store(s) 1240 that can be employed to store information local to the servers 1230.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2(3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
at least one memory that stores computer-executable components; and
at least one processor that executes the computer-executable components stored in the at least one memory, wherein the computer-executable components comprise:
a similarity mapping component that performs a similarity mapping process between pairs of medical imaging protocol data, the pairs respectively comprising planned imaging protocol data in a first format and performed imaging protocol data in a second format, and generates similarity matrices for the pairs as a result of the similarity mapping process; and
a training component that trains a neural network model to estimate similarity scores for the pairs using the similarity matrices as input to the neural network model.

2. The system of claim 1, wherein the training component trains the neural network model using a supervised machine learning process and ground truth information for respective ones of the pairs identifying ground truth similarity scores.

3. The system of claim 1, wherein the similarity mapping process comprises, for each pair, determining measures of similarity between respective parameters of a defined set of parameters included in the planned imaging protocol data and the performed imaging protocol data, and generating a similarity matrix for the pair comprising the measures of similarity.

4. The system of claim 3, wherein the defined set of parameters comprises one or more parameters selected from the group consisting of: protocol name, series description name, anatomic region, series duration, field of view, slice thickness, percent phase field of view, number of excitations, acceleration factor, specific absorption rate, echo time, receive coil name, pixel bandwidth, repetition time, echo train length and voxel size.

5. The system of claim 3, wherein determining the measures of similarity comprises determining the measures of similarity based on correspondences and differences between terms of the respective parameters in the first format and the second format.

6. The system of claim 1, wherein the computer-executable components further comprise:
an inferencing component that employs a trained version of the neural network model to estimate similarity scores for new pairs of medical imaging protocol data based on new similarity matrices generated for the new pairs by the similarity mapping component using the similarity mapping process, wherein the new pairs respectively comprise new planned imaging protocol data in the first format and new performed imaging protocol data in the second format.

7. The system of claim 6, wherein the computer-executable components further comprise:
a performance assessment component that identifies a subset of the new pairs with similarity scores below a threshold similarity score, and determines, for respective pairs of the subset, whether one or more performance metrics for imaging scans of the subset satisfy one or more acceptability criterion.

8. The system of claim 7, wherein the one or more performance metrics comprise a quality metric that represents a measure of quality of medical images acquired via the imaging scans.

9. The system of claim 7, wherein the one or more performance metrics comprise a dose metric that represents a measure of radiation dose associated with the imaging scans.

10. The system of claim 6, wherein the computer-executable components further comprise:
a performance assessment component that identifies a subset of the new pairs with similarity scores above a threshold similarity score, and determines, for respective pairs of the subset, whether one or more performance metrics for imaging scans of the subset satisfy one or more acceptability criterion.

11. The system of claim 10, wherein the one or more performance metrics comprise a quality metric that represents a measure of quality of medical images acquired via the imaging scans.

12. The system of claim 10, wherein the one or more performance metrics comprise a dose metric that represents a measure of radiation dose associated with the imaging scans.

13. A method, comprising:
performing, by a system comprising a processor, a similarity mapping process between pairs of medical imaging protocol data, the pairs respectively comprising planned imaging protocol data in a first format and performed imaging protocol data in a second format;
generating, by the system, similarity matrices for the pairs as a result of the similarity mapping process; and
training, by the system, a neural network model to estimate similarity scores for the pairs using the similarity matrices as input to the neural network model.

14. The method of claim 13, wherein the training comprises training the neural network model using a supervised machine learning process and ground truth information for respective ones of the pairs identifying ground truth similarity scores.

15. The method of claim 13, wherein the similarity mapping process comprises, for each pair, determining measures of similarity between respective parameters of a defined set of parameters included in the planned imaging protocol data and the performed imaging protocol data, and generating a similarity matrix for the pair comprising the measures of similarity.

16. The method of claim 15, wherein the defined set of parameters comprises one or more parameters selected from the group consisting of: protocol name, series description name, anatomic region, series duration, field of view, slice thickness, percent phase field of view, number of excitations, acceleration factor, specific absorption rate, echo time, receive coil name, pixel bandwidth, repetition time, echo train length and voxel size.

17. The method of claim 15, wherein determining the measures of similarity comprises determining the measures of similarity based on correspondences and differences between terms of the respective parameters in the first format and the second format.

18. The method of claim 13, further comprising:
employing, by the system, a trained version of the neural network model to estimate similarity scores for new pairs of medical imaging protocol data based on new similarity matrices generated for the new pairs by the system using the similarity mapping process, wherein the new pairs respectively comprise new planned imaging protocol data in the first format and new performed imaging protocol data in the second format.

19. The method of claim 18, further comprising:
identifying, by the system, a subset of the new pairs with similarity scores below a threshold similarity score;
determining, by the system, for respective pairs of the subset, differences between one or more reference performance metrics for imaging scans that adhere to the new planned imaging protocol data and actual performance metrics for performed imaging scans of the subset that adhere to the new performed imaging protocol data;
identifying, by the system, one or more pairs of the subset associated with differences that correspond to an improvement to the one or more performance metrics; and
determining, by the system, a modification to the new planned imaging protocol data that facilitates the improvement based on the new performed imaging protocol data for the one or more pairs.

20. A non-transitory machine-readable storage medium, comprising executable instructions that, when executed by a processor, facilitate performance of operations, comprising:
performing a similarity mapping process between pairs of medical imaging protocol data, the pairs respectively comprising planned imaging protocol data in a first format and performed imaging protocol data in a second format;
generating similarity matrices for the pairs as a result of the similarity mapping process; and
training a neural network model to estimate similarity scores for the pairs using the similarity matrices as input to the neural network model.
